(19) United States
(12) Patent Application Publication
(10) Pub. No.: US 2004/0187169 A1
(43) Pub. Date: Sep. 23, 2004

(54) Method of screening for genes or agents affecting the response of the Drosophila heart to hypoxia or anoxia

(76) Inventor: Giovanni Paternostro, Del Mar, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/804,645

(22) Filed: Mar. 19, 2004

Related U.S. Application Data
(60) Provisional application No. 60/456,846, filed on Mar. 21, 2003.

(51) Int. Cl. A01K 67/033

(52) U.S. Cl. 800/3; 800/8

(57) ABSTRACT

The present invention provides a novel method of screening for a gene or an agent affecting cardiac function after or during hypoxia or anoxia, including the steps of: providing an adult Drosophila in conditions able to induce hypoxia or anoxia, imaging the heart of the Drosophila, measuring the movements of the heart in the image, analyzing the measurements of the movements, and identifying a gene affecting the cardiac function of the Drosophila or identifying the effect of the agent on the cardiac function. The method may include administering an agent before, during or after hypoxia.

### CROSS REFERENCE TO RELATED APPLICATIONS

This application claims benefit of priority to U.S. Provisional Patent Application No. 60/456,846 filed on Mar. 21, 2003 entitled Method for Screening for Genes or Agents Affecting the Response of the Drosophila Heart to Hypoxia or Anoxia and naming Giovanni Paternostro as inventor, which is herein incorporated by reference in its entirety.

### TECHNICAL FIELD

The present invention relates generally to methods of screening for compounds or nucleic acids and more particularly to a method for screening for a compound, protein or nucleic acid, such as a gene, able to affect cardiac function during or after hypoxia or anoxia.

### BACKGROUND OF THE INVENTION

Drosophila as a Model for Human Heart Disease

The genome of Drosophila melanogaster was the first to be fully sequenced for an animal possessing a circulatory system (4). The heart of the fly consists of a tubular structure that contracts spontaneously throughout the insect's lifespan and has the main function of circulating the hemolymph which transports energy substrates from the abdomen to the thorax and head (5). The normal lifespan of the fruitfly depends on the temperature at which the flies are kept, being shorter at higher temperatures. The mean lifespan of D. melanogaster is 45-60 days at 25° C. (6).

Several groups have exploited Drosophila genetics for identifying genes regulating cardiac development in the fly, and this approach has proved to be useful for guiding research on cardiac development in vertebrates. One of the more notable examples is the identification of the Drosophila gene tinman (7), which prompted the cloning of homologues regulating cardiac development in mice (Nkx2-5/Csx) (8, 9). The finding of homologous genes that similarly influence development of the heart-like organ of Drosophila and the mouse heart suggests that at least some aspects of fly cardiac biology are common to mammals. The relevance of some fly genes to human cardiac pathology is also supported by the finding that mutations in the HERG potassium channel gene cause long-QT syndrome, a potentially fatal cardiac arrhythmia (10). HERG stands for “human ether-a-go-go related gene” and it was first identified by virtue of its homology to the Drosophila potassium channel gene “ether-a-go-go” (11).

Several human disease models have been developed in Drosophila, particularly for neurological diseases (12-14). Drosophila is also commonly employed as a model-organism for studying the genetics of aging, partly because it represents a genetically tractable organism with a short life span (15). For example, genetic screens have allowed the identification of a single gene that controls life span in flies, increasing it by ≈35% (16). However very little is known about the cardiac changes that occur with hypoxia in the fly.

Our recent paper was the first attempt to exploit Drosophila melanogaster for investigations of adult cardiac dysfunction (2, 17). We developed methods for studying cardiac function in vivo in adult flies. Using 2 different cardiovascular stress methods (elevated ambient temperature and external electrical pacing), we found that maximal heart rate is significantly and reproducibly reduced with aging in Drosophila, analogous to observations in elderly humans (18). We also described for the first time several other aspects of the cardiac physiology of young adult and aging Drosophila, including an age-associated increase in rhythm disturbances.

Molecular Basis of Hypoxic Damage in the Heart

The expression of a large number of genes is altered during hypoxia and reoxygenation of the heart (19). However the exact mechanism whereby reversible cell damage finally evolves into irreversible infarction is still controversial (20).

Loss of ATP will initially stimulate anaerobic glycolysis but the consequent decrease in pH will lead to its inhibition (20). In ischemia, pH also decreases as a consequence of reduced washout of metabolically produced CO₂ (20). Glycolytically produced ATP has been proposed to be essential for membrane-associated ion pumps (21, 22). Inhibition of the sodium pump might lead to increase in intracellular osmotic pressure and irreversible membrane damage (23).

Altered calcium homeostasis and an increase in intracellular calcium are also consequences of decreased uptake by the sarcoplasmic reticulum and decreased extrusion from the cell resulting from lack of ATP (24). Calcium accumulates in the mitochondria, leading to mitochondrial damage. Damaged mitochondria upon reoxygenation will generate free oxygen radicals (25). It has been proposed that oxygen radicals can cause membrane peroxidation and lead to cell death (26). Antioxidant therapies have not however consistently been shown to be beneficial in this setting (27).

Another proposed mechanism of cell death in the hypoxic myocardium is related to the activation of apoptosis proteins (28, 29), especially at the time of reperfusion/reoxygenation. Hypoxia has been reported to cause the activation of proteolytic enzymes in cardiac myocytes: caspases in some models (30) and calpains in others (31). Other authors report that apoptosis is linked either to the decrease in pH during hypoxia or to reoxygenation (32). In adult cardiomyocytes both caspase inhibition and over-expression of the anti-apoptotic protein Bcl-2 can improve cellular viability during reoxygenation but has minimal effect on hypoxia induced cell death (33).

In conclusion, at present there is not a clear understanding of the relative importance of these factors and of the best way to intervene to protect the heart from hypoxic damage (20).

Aging and Cardiac Hypoxia

The relation between aging and heart disease is clear (34). The prevalence of heart failure is almost 70 times higher in persons 65 years of age or older, compared to persons aged 20-34 years (34). Nearly 80% of hospital admissions in the United States for heart failure involve patients over 65 years of age (35). Ischemic heart disease is the main cause of cardiac failure (34) and tobacco smoke contributes greatly to this disease burden (36).

Several papers have shown that hypoxic damage is more severe in older hearts. Contractile recovery is reduced (37, 38) in rat hearts after hypoxia and reoxygenation. Several of the known mediators of hypoxic damage are also altered by cardiac aging. For example, the increase in intracellular calcium is exacerbated and the generation of intracellular reactive oxygen species is enhanced (39). This has been attributed to alteration in the expression of the proteins that regulate calcium handling and to mitochondrial dysfunction in the aging heart (40). This reduced tolerance to hypoxia of the aged myocardium has been confirmed in studies of human atrial trabecule harvested during cardiac surgery (41).

Cardiac hypoxia interacts with the changes caused by aging on the heart and the short life-span of Drosophila makes the study of the genetics of this important interaction possible.

### SUMMARY

The present invention includes a method of screening for a nucleic acid such as a gene affecting cardiac function after or during hypoxia or anoxia, including the steps of: exposing an adult Drosophila to conditions able to induce cardiac hypoxia or anoxia, imaging the heart of the Drosophila, measuring the movements of the heart in the image, analyzing the measurements of the movements, and identifying a gene affecting the cardiac function of the Drosophila. A gene is identified from Drosophila having measurements different than control Drosophila. The analysis of the measurements are indicative of the cardiac function of the Drosophila and changes in the function are indicative of the effect of the gene on the cardiac function of the Drosophila after or during cardiac hypoxia or anoxia.

A second aspect of the invention includes a method of screening for agents affecting cardiac function after or during hypoxia or anoxia, including the steps of: exposing an adult Drosophila to conditions able to induce cardiac hypoxia or anoxia, exposing the Drosophila to an agent, imaging the heart of the Drosophila, measuring the movements of the heart in the image, analyzing the measurements of the movements, and identifying an effect of the agent on the cardiac function of the Drosophila by comparing the analysis to a control. The analysis of the measurements are indicative of the cardiac function of the Drosophila and changes in the function are indicative of the effect of the agent on the cardiac function of the Drosophila after or during cardiac hypoxia or anoxia.

A third aspect of the present invention includes identifying a nucleic acid such as a gene, a compound or agent able to affect, mediate, prevent or protect against age-related changes that correlate with an increased risk of cardiac hypoxia or anoxia using a disclosed method of screening for a nucleic acid or an agent.

### BRIEF DESCRIPTION OF THE DRAWINGS

FIG. 1 depicts a graphical representation of heart movement measurements obtained with the image analysis software. Specifically, the graph depicts heart contraction recovery after 18 hours of hypoxia at 18° C. in young (1-day-old, depicted as triangles) and aged (30-day-old, depicted as squares) wild-type Oregon-R male flies. The recovery is impaired in older flies. The older flies exhibit a phenotype similar to that of young mutants with increased cardiac damage after hypoxia Data are mean±SEM, n=6 in both groups.

### DETAILED DESCRIPTION

The present invention addresses the shortcomings in current screening methods and provides a novel method of screening for a nucleic acid such as a gene affecting cardiac function after or during hypoxia or anoxia, including the steps of providing an adult Drosophila in conditions able to induce hypoxia or anoxia, imaging the heart of the Drosophila, measuring the movements of the heart in the image, and analyzing the measurements of the movements. Analysis of the measurements are indicative of the cardiac function of the Drosophila and changes in the function may be indicative of the effect of the gene on the cardiac function of the Drosophila after or during cardiac hypoxia or anoxia.

Drosophila has a hypoxic response pathway analogous to that of mammals: it has a functional homologue of HIF (hypoxia-inducible factor), called Sima, and of other members of this pathway (42, 43). HIF and Sima are transcription factors that accumulate under hypoxic conditions and activate a similar set of genes, e.g. glycolytic enzymes (43). Other mediators of the hypoxic response in mammals, nitric oxide and protein kinase G, are also components of the response to hypoxia in Drosophila (44).

The only published Drosophila mutant screen for hypoxia response genes has been performed by the group of Haddad (45, 46). This was a screen for loss-of-function mutants, which exhibited retarded recovery of mobility after hypoxia. It was shown that the phenotype was mainly dependent on the effect of hypoxia on the nervous system (47). Hypnos-2, a gene expressed in nervous system and involved in RNA editing, was identified. The product of this gene targets several ion channels (48).

As shown herein, the response of the heart to hypoxia in Drosophila differs from that of nerve cells, justifying a separate screen. This is consistent with the differences between these two tissues in many of the proposed molecular mechanisms of hypoxic damage, e.g. differences in calcium handling or in the relative fluxes of metabolic pathways. Even different parts of the central nervous system seem to differ greatly in their response to oxygen deprivation (49).

The present invention includes the use of a Drosophila model to identify nucleic acids, genes, proteins, compounds or agents that effect cardiac function. There are a number of advantages to a Drosophila melanogaster model. These advantages include, but are not limited to a large number of genetic screens can be performed, genetic interactions can be identified by crossing the mutant of interest with collections of other mutants which among other benefits facilitates the identification of modifier genes such as suppressors or enhancers, the short life-span facilitates the study of the effects of aging, many mutants and chromosomal markers are available, there are a number of known genetic techniques that are powerful and have been perfected since the beginning of the last century and the genome has been sequenced, thereby greatly speeding efforts to identify the genes responsible for mutant phenotypes.

Many important findings for human medicine and biology have originated from studies in Drosophila or other invertebrates. Examples are the identification of genes regulating embryonal development in Drosophila (51), the initial identification of several components of the apoptotic machinery (52) and elucidation of gene pathways involved in neurogenesis (53). In all these examples, rapid progress in the understanding of complex problems was made possible by initial investigations in Drosophila and subsequent extensions of the findings to mammals and humans. Given that recent surveys have shown remarkable conservation of human genes in the fly genome, including cardiac disease-relevant genes (54, 55), genes and agents identified by our studies have a strong possibility of being relevant to humans.

The present invention includes but is not limited to the testing of mutant Drosophila. Mutants may be prepared using a variety of techniques known in the molecular biology, biochemistry and chemical arts as well as their applications in the arts pertaining to Drosophila genetics. As a non-limiting example, mutants can be obtained using chemical mutagenesis or by using transposon insertions or deletions (see St Johnston, D. 2002. The art and design of genetic screens: Drosophila melanogaster Nat Rev Genet, 3: 176-88). A Mutation may result in a change in expression of a gene which affects cardiac function.

The present invention includes a variety of non-limiting methods of inducing hypoxia or anoxia in Drosophila. These methods may include but are not limited to administering gaseous carbon dioxide (CO₂) or nitrogen (N₂) in an amount sufficient to induce hypoxia in a typical or normal Drosophila. As general guidance, hypoxia may be induced after about 2 hours of incubation in CO₂ and about 5 hours of incubation in N₂. Hypoxia may be studied by incubating Drosophila at elevated or reduced temperatures, such as those that are higher or lower than room temperature.

The present invention includes imaging the heart of the Drosophila and identifying Drosophila with cardiac images different than that of a control Drosophila. The disclosed methods are performed by detecting and analyzing images of heart movement, such as movement of the heart walls by using movement detection software. Any software able to detect, record or analyze images of a beating heart in Drosophila may be used with the present invention.

As will be demonstrated by a variety of non-limiting examples, imaging may be performed using a variety of techniques. In one embodiment the Drosophila is positioned under a microscope so that the light beam of the microscope is generally perpendicular to the frontal plane of the Drosophila and is directed on the heart of the Drosophila. The images are then recorded by a suitable recording means such as a computer equipped with appropriate recording software or by using high-speed photography. The quality of the images may be enhanced or adjusted using one or more contrast enhancing means located on the microscope or on recording or analysis software.

In another embodiment, heart movement is imaged by the use of an expressed fluorescent protein. In this embodiment the Drosophila expresses a fluorescent protein in the heart to enhance the imaging. One example of a fluorescent protein commonly used in the life science industry green fluorescent protein (GFP). GFP has been selectively expressed in a variety of organs and may be expressed in the heart. Detection may be performed using a microscope having fluorescent detection capabilities, of which many are available in the art.

Drosophila that vary from a control or control set of data may be identified as candidates for further studies. Further studies may include the identification of a nucleic acid, a gene, an enhancer or suppressor able to directly or indirectly, such as by the expression of a cofactor, affect cardiac function. For example, for mutants that are associated with P element insertions, we can confirm that the mutant phenotype is a direct consequence of the element insertion in or nearby a specific gene. The first step can be to identify and confirm the DNA sequences flanking P-element insertions, in P element mutants, by using inverse PCR. Confirmation that the P insertion is responsible for the mutant phenotype can be obtained by showing that precise excision of the P element reverts the phenotype to wild type.

A gene can be expressed or overexpressed in Drosophila either in the whole body or specifically in the heart using a cardiac specific promoter. We can also use the GAL4/UAS system, in which two constructs are introduced in the same fly by appropriate crosses, or inducible promoters as the tet-on system, in which tetracyline regulates the expression of a gene. Our method can be used to identify any protective effects of this gene against cardiac damage during or after hypoxia or anoxia

The present invention may have therapeutic or diagnostic benefits in the treatment of human conditions such as hypoxia or anoxia. A nucleic acid such as a gene, identified using the disclosed methods may be targeted or used in the treatment of hypoxia or anoxia. As a non-limiting example, such treatment may include administering a therapeutically effective dose of a compound able to affect transcription or translation of the nucleic acid to prevent, protect against, reduce or mediate age-related changes in older hearts and thereby decrease the occurrence of hypoxic damage. These age-related changes may directly or indirectly affect the handling of intracellular calcium. To further examine the therapeutic or diagnostic capabilities of targets identified using the present methods, nucleic acid sequences may be tested in additional in vivo models such as mouse, rat, rabbit and human or in perfused heart preparations in vertebrate models.

The present invention also includes a method of screening for agents affecting cardiac function after or during hypoxia or anoxia, including the steps of exposing an adult Drosophila to conditions able to induce cardiac hypoxia or anoxia, exposing the Drosophila to an agent, imaging the heart of the Drosophila, measuring the movements of the heart in the image, analyzing the measurements of the movements, and identifying an effect of the agent on the cardiac function of the Drosophila by comparing the analysis to a control.

The present invention recognizes a variety of or agents or compounds may be screened for therapeutic or diagnostic use against hypoxia or anoxia. Specifically a compound may be administered prior, during or after inducing hypoxia in Drosophila. The compound may be in a variety of states or administered in a variety of techniques such as an aerosol, in the food or in the water.

The present invention may identify a compound or agent able to affect, mediate, prevent or protect against age-related changes in an individual that lead to an increased risk of hypoxia or anoxia. The present invention may identify compounds or agents that affect age-related changes by altering the handling of intracellular calcium. A compound or agent identified using the disclosed screening methods may be undergo further testing by its in vivo effect in additional animal models such as but not limited to mouse, rat, rabbit or human models or in perfused heart preparations in vertebrate models.

### EXAMPLE 1

### Overview of the Drosophila Heart Imaging System and Mutants Used in a Study

Flies are mounted on glass slides and observed with a Nikon Diaphot-TMD inverted microscope, with Nomarski (DIC) optics and a 10× (N.A. 0.25) objective. Images are obtained by closing the diaphragm, so that the light-beam is concentrated on the first ventricle of the heart. Flies are positioned on their backs, exactly perpendicular to the light path, and fixed in this position by mounting the wings on the glass slide with double-stick tape.

An account of our Drosophila heart imaging system has been published (2). In our initial publication we have shown differences in cardiac function between young and aged fly hearts that mimic those observed in humans.

We have now performed a pilot hypoxia screen on 700 mutants, with the aim of identifying a first set of potentially informative mutants and of optimizing our protocol. The mutants were obtained by P element insertion and most of them were part of the Drosophila gene disruption project (56).

We have improved the hardware of our set-up, obtaining better reproducibility. We use glass vials to minimize gas exchange and we add absorbent paper wetted with 100 microliter of water to obtain comparable humidity in each vial. Flow and duration of replacement with gases are kept exactly constant.

### EXAMPLE 2

### Identifying Drosophila with Increased Resistance to Hypoxia

Hypoxia was achieved using CO₂ in 350 fly stocks and nitrogen in the remaining 350. As expected, CO₂ was more damaging for the fly heart. Flies do have carbonic anhydrase and an increase in CO₂ lowers the pH more than simple hypoxia. Changes in pH are in important component of ischemic damage (57). As a comparison, at the temperature of 32° C., the same amount of cardiac damage was obtained in wild type Oregon flies after 2 hours of incubation in CO₂ and after 5 hours of incubation in nitrogen. From the 350 mutants screened using CO₂ we isolated 35 mutants with improved cardiac resistance to hypoxia. These were also tested with nitrogen, and no significant correlation was found between the response to the two gases (the pH-related damage might be affected by a specific set of genes). We have, however, isolated 6 mutants that seem to be resistant to both gases.

In the screen of the 350 mutants where we used nitrogen we identified 13 mutants with reproducible increased cardiac resistance to hypoxia. The genetic background can have a large effect on some complex Drosophila phenotypes, for example behavioral phenotypes (58). We also identified 39 mutants that exhibit reduced cardiac recovery after hypoxia.

### EXAMPLE 3

### Assessment of Cardiac Damage After Hypoxia

During the pilot screen we progressively refined our methods of assessment of cardiac damage. The more convenient duration of this period depends on the temperature at which the flies are kept, cardiac damage being much faster at higher temperatures. This is consistent with the finding that metabolic rate depends on environmental temperature in flies, which are poikiloterms (59) Therefore increased temperature would exacerbate ATP depletion during hypoxia. For example, a comparable amount of damage after incubation with nitrogen (corresponding to a recovery of cardiac contraction in 30% of 2-4 days old wild type Oregon flies) is obtained after 5 hours at 32° C. and after 18 hours at 18° C.

Similarly to what is observed in the human heart using echocardiography after ischemia (60), there can be, however, more subtle changes in cardiac function in the Drosophila heart after hypoxia. The amplitude of cardiac contraction can be reduced or the heart wall can contract in a very irregular way. These cardiac phenotypes are called hypokinesia and dyskinesia by human echocardiographers (60). We could not, therefore, easily categorize some hearts as beating normally or not beating and developed a scoring system with 5 levels of increasing severity to measure the effect of hypoxia. We have scored independently a great number of hearts and our scores appear to be reproducible and consistent.

### EXAMPLE 4

### Measuring and Analyzing the Measurements of Drosophila Using a Software Loaded Computer

In addition, a dedicated image analysis software was recently written to rapidly quantify the amount of contraction of the heart. The measurements are obtained in less than 30 seconds. The computer program analyzes images of the fly heart obtained using a DT3155 PCI frame grabber (Data Translation, Marlboro Mass.) operating in conjunction with a camera (Sony DXC 101) and microscope. Images are processed, and a readout is given of the percentage of sampled pixels that change intensity, which correlates with the degree of movement in the sampled images. The main algorithm is simply a comparison of pixel intensity across different frames. For each pixel sampled, we analyze two frames in order to compare pixel intensity. First the pixel intensity in each frame is quantified, then subtracted. If the result after subtraction is zero, then the pixel will not be counted as changed. This is repeated across every pair of frames in the acquisition. Several sensitivity parameters were optimized to obtain a good signal to noise ratio.

The goal in creating the Movement detection software is to obtain a computer automated system by which heart wall movement in various fruit flies can be studied without human bias. The basic concept behind the software is to analyze pixel intensities in a 40×40 pixel box in the center of a series of 640×480 resolution images. Obtained using a DT3155 PCI frame grabber operating in conjunction with a camera and microscope, these images are processed and a program readout is given that details the number and percentage of sampled pixels that changed intensity, which correlates to degree of movement in the sampled images.

The main algorithm at its core is simply a comparison of pixel intensity across different frames. For each pixel sampled, we must analyze two frames in order to compare pixel intensity. First the pixel intensity in each frame is quantified, then subtracted. In theory, if the result after subtraction is zero, then the pixel will not be counted as changed. This is repeated across every pair of frames in the acquisition.

Four main parameters can be varied by the user for each acquisition: acquire time(s); tolerance (pixels); number of lines acquired; and acquire width, all of which will be explained shortly. The acquire time is simply the number of seconds of video that the user wishes to span in the analysis. Each second corresponds to 30 frames, as 30 fps is the rate of operation of the frame grabber. Clearly, the longer the period of the acquisition, the more reliable the data that can be obtained. Tolerance is the amount of intensity variation a pixel is allowed without being classified as changed; this was included as a parameter to help deal with noise that could be produced by the various devices between the camera and the software. The third parameter is the number of evenly spaced 40 pixel wide lines to acquire in the 40×40 box, and can be maximized at 40. By acquiring more lines, a more accurate analysis can be obtained, albeit at a slower rate. Every pixel in each of the lines acquired is sampled for change. The fourth parameter, which we have referred to as acquire width, enables the user to select which frames to analyze. To illustrate how this works, by setting it at 2, we pair up frames 1 and 2, 3 and 4, 5 and 6, 7 and 8, etc. and subtract them in order to determine movement (takes frames in groups of 2 for analysis). Setting acquire width at 3 pairs up frames 1 and 3, 4 and 6, 7 and 9, 10 and 12, etc. for subtraction in order to determine movement (takes frames in groups of 3, skips the second of each group).

Currently, there are two different versions of this software, which are identical except for the treatment of the last parameter, acquire width. The first program treats the fourth parameter as described above, in that it is considered variable, and the user can effectively select which frames he wants to analyze. In this first program it was found that an acquire width of 3 produced optimal results. The second program uses an alternative method of frame processing, known as the double difference method (as opposed to the single difference method used in the first program). With double difference, we sample four instead of two frames, subtracting the first two, obtaining two results, and then subtracting the two results. If this final result is higher than the tolerance, then the pixel is counted as changed. As this program was developed after the first, we used a fixed acquire width of 3, such that the four frames we were acquiring were the first and third in two groups of 3 frames (for example, 1, 3, 4, and 6).

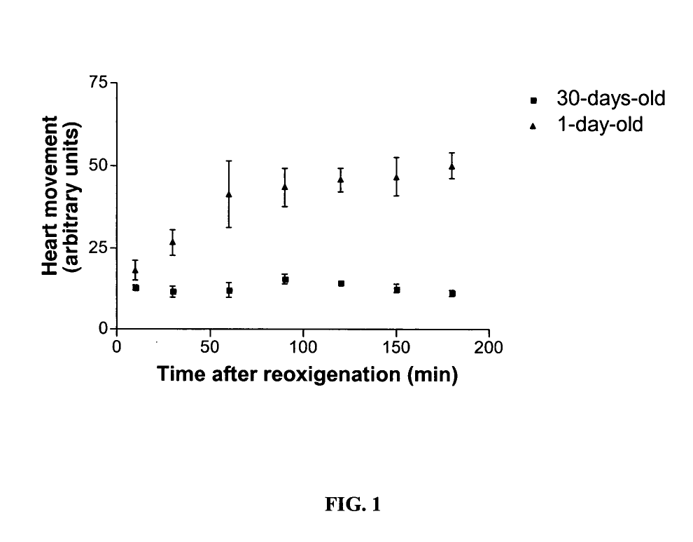

Referring to FIG. 1 we show an example of the results obtained with the image analysis software or Movement detection software in an experiment comparing the recovery from 18 hours of hypoxia at 18° C. of 1-day-old and 30-days-old flies (these ages, and all others given in the application, are expressed as days after the eclosion of adult flies from the pupal cage). The decreased recovery after hypoxia of the older fly heart has been confirmed using visual scoring in several other experiments, with 30-days old flies. Furthermore, in all files studied with this protocol at 60 days of age (n=30) the heart was completely still 1 hour after reoxigenation. This is consistent with what has been reported in the hearts of rodents and humans (39, 41).

The time course shown in FIG. 1 is representative of those obtained in all other experiments we performed with visual scoring using the same protocol. Cardiac recovery is close to maximum after about one hour and stays close to these levels until at least the third hour after the cessation of hypoxia. As a rapid screening method, we intend to use the software to obtain a single measurement 2 hours after reoxigenation. With this protocol we intend to measure the maximum recovery of function after severe hypoxia.

### EXAMPLE 5

### The Use of High Speed Photography to Confirm Changes in Cardiac Function

We can also measure the temporary impairment of cardiac contraction after hypoxia of shorter duration. Using a high-speed camera (Motionscope PCI, Redlake, capable of obtaining up to 1000 frames per second) and another specially written image analysis program we can obtain an M-mode image. The M-mode image (mono-dimensional image) is a time-space image signal representing the time course of image intensity along a line segment that crosses the ventricular lumen transverse to the heart axis. We use this term because it is commonly used in clinical echocardiography. From the M-mode image we can accurately measure systolic and diastolic dimensions, the duration of systole and diastole and of their rapid and slow phases and we can estimate ejection fraction. The camera with high frame rates is necessary because an ordinary camera records only 30 frames per second, which would be equivalent to only 6 frames in the entire cardiac cycle of a fly heart beating at 5 Hertz, allowing only limited accuracy. We observe temporary impairment of cardiac wall motion and reduced ejection fraction in the first few minutes after 2 hours of hypoxia. This is similar to what has been reported in the human heart, using echocardiography, during and immediately after transient ischemia (60).

The recovery of general body mobility was always much delayed compared to the resumption of heart beat after hypoxia. For example after 2 hours of hypoxia (nitrogen) at 22° C. the heart beat recovered within 5 minutes in all flies but the average time before any spontaneous body movement could be detected was 25±2 minutes (n=10). The recovery of body movement most likely represent progressive recovery of neurological function as shown by the group of Haddad using electrophysiological recordings in giant fiber systems neurons (47, 61) after nitrogen exposure. Indeed the gene they identified by screening for delayed recovery of body mobility is mainly expressed in the nervous system (48). These results suggest that the heart and the nervous system are differently affected by hypoxia. The delayed recovery of body movement is convenient for our purposes because heart function can be studied without anesthesia under these conditions.

### REFERENCES

1. Smith, C. J., and Fischer, T. H. 2001. Particulate and vapor phase constituents of cigarette mainstream smoke and risk of myocardial infarction. Atherosclerosis 158:257-267.

2. Paternostro, G., Vignola, C., Bartsch, D. U., Omens, J. H., McCulloch, A. D., and Reed, J. C. 2001. Age-associated cardiac dysfunction in Drosophila melanogaster. Circulation Research 88:1053-1058.

3. Gray, I. C., Campbell, D. A., and Spurr, N. K. 2000. Single nucleotide polymorphisms as tools in human genetics. Hum Mol Genet 9:2403-2408.

4. Adams, M. D., Celniker, S. E., Holt, R. A., Evans, C. A., Gocayne, J. D., Amanatides, P. G., Scherer, S. E., Li, P. W., Hoskins, R. A., Galle, R. F., et al. 2000. The genome sequence of Drosophila melanogaster. Science 287:2185-2195.

5. Rizki, T. M. 1978. The Circulatory System and Associated Cells and Tissues. In The Genetics and Biology of Drosophila. M. Ashburner, and T. R. F. Wright, editors. London: Academic Press. 397-452.

6. Ashburner, M. 1989. Drosophila. A laboratory handbook. Cold Spring Harbor: Cold Spring Harbor Laboratory Press.

7. Bodmer, R., and Venkatesh, T. V. 1998. Heart development in Drosophila and vertebrates: conservation of molecular mechanisms. Developmental Genetics 22:181-186.

8. Lints, T. J., Parsons, L. M., Hartley, L., Lyons, I., and Harvey, R. P. 1993. Nkx-2.5:

a novel murine homeobox gene expressed in early heart progenitor cells and their myogenic descendants. Development 119:419-431.

9. Komuro, I., and Izumo, S. 1993. Csx: a murine homeobox-containing gene specifically expressed in the developing heart. Proceedings of the National Academy of Sciences of the United States of America 90:8145-8149.

10. Curran, M. E., Splawski, I., Timothy, K. W., Vincent, G. M., Green, E. D., and Keating, M. T. 1995. A molecular basis for cardiac arrhythmia: HERG mutations cause long QT syndrome. Cell 80:795-803.

11. Warmke, J. W., and Ganetzky, B. 1994. A family of potassium channel genes related to eag in Drosophila and mammals. Proc Natl Acad Sci USA 91:3438-3442.

12. Min, K. T., and Benzer, S. 1999. Preventing neurodegeneration in the Drosophila mutant bubblegum. Science 284:1985-1988.

13. Kazemi-Esfaijani, P., and Benzer, S. 2000. Genetic suppression of polyglutamine toxicity in Drosophila. Science 287:1837-1840.

14. Feany, M. B., and Bender, W. W. 2000. A Drosophila model of Parkinson's disease. Nature 404:394-398.

15. Rose, M. R. 1991. Evolutionary Biology of Aging. New York: Oxford University Press.

16. Lin, Y. J., Seroude, L., and Benzer, S. 1998. Extended life-span and stress resistance in the Drosophila mutant methuselah. Science 282:943-946.

17. Lakatta, E. G. 2001. Heart aging: a fly in the ointment? Circulation Research 88:984-986.

18. Fleg, J. L., O'Connor, F., Gerstenblith, G., Becker, L. C., Clulow, J., Schulman, S. P., and Lakatta, E. G. 1995. Impact of age on the cardiovascular response to dynamic upright exercise in healthy men and women. Journal of Applied Physiology 78:890-900.

19. Piacentini, L., and Karliner, J. S. 1999. Altered gene expression during hypoxia and reoxygenation of the heart. Pharmacol Ther 83:21-37.

20. Opie, L. H. 1998. The Heart. New York: Raven Press.

21. Weiss, J., and Hiltbrand, B. 1985. Functional compartmentation of glycolytic versus oxidative metabolism in isolated rabbit heart. J Clin Invest 75:436-447.

22. Cross, H. R., Radda, G. K., and Clarke, K. 1995. The role of Na+/K+ ATPase activity during low flow ischemia in preventing myocardial injury: a 31 P, 23Na and 87Rb NMR spectroscopic study. Magn Reson Med 34:673-685.

23. Jennings, R. B., Reimer, K. A., and Steenbergen, C. 1986. Myocardial ischemia revisited. The osmolar load, membrane damage, and reperfusion. J Mol Cell Cardiol 18:769-780.

24. Steenbergen, C., Murphy, E., Levy, L., and London, R. E. 1987. Elevation in cytosolic free calcium concentration early in myocardial ischemia in perfused rat heart. Circ Res 60:700-707.

25. Ambrosio, G., Weisfeldt, M. L., Jacobus, W. E., and Flaherty, J. T. 1987. Evidence for a reversible oxygen radical-mediated component of reperfusion injury: reduction by recombinant human superoxide dismutase administered at the time of reflow. Circulation 75:282-291.

26. Meerson, F. Z., Kagan, V. E., Kozlov Yu, P., Belkina, L. M., and Arkhipenko Yu, V. 1982. The role of lipid peroxidation in pathogenesis of ischemic damage and the antioxidant protection of the heart. Basic Res Cardiol 77:465-485.

27. Kloner, R. A., Przyklenk, K., and Whittaker, P. 1989. Deleterious effects of oxygen radicals in ischemia/reperfusion. Resolved and unresolved issues. Circulation 80:1115-1127.

28. Gottlieb, R. A., Burleson, K. O., Kloner, R. A., Babior, B. M., and Engler, R. L. 1994. Reperfusion injury induces apoptosis in rabbit cardiomyocytes. J-Clin-Invest 94:1621-1628.

29. Fliss, H., and Gattinger, D. 1996. Apoptosis in ischemic and reperfused rat myocardium. Circulation Research 79:949-956.

30. de Moissac, D., Gurevich, R. M., Zheng, H., Singal, P. K., and Kirshenbaum, L. A. 2000. Caspase activation and mitochondrial cytochrome C release during hypoxia-mediated apoptosis of adult ventricular myocytes. J Mol Cell Cardiol 32:53-63.

31. Chen, M., He, H., Zhan, S., Krajewski, S., Reed, J. C., and Gottlieb, R. A. 2001. Bid is cleaved by calpain to an active fragment in vitro and during myocardial ischemia/reperfusion. J Biol Chem 276:30724-30728.

32. Webster, K. A., Discher, D. J., Kaiser, S., Hernandez, O., Sato, B., and Bishopric, N. H. 1999. Hypoxia-activated apoptosis of cardiac myocytes requires reoxygenation or a pH shift and is independent of p53. J Clin Invest 104:239-252.

33. Kang, P. M., Haunstetter, A., Aoki, H., Usheva, A., and Izumo, S. 2000. Morphological and molecular characterization of adult cardiomyocyte apoptosis during hypoxia and reoxygenation. Circ Res 87:118-125.

34. AHA. 1999. 2000 Heart and Stroke Statistical Update. Dallas, Tex: American Heart Association.

35. Rich, M. W. 1999. Heart failure. In Principles of geriatric medicine and gerontology. W. R. Hazzard, J. P. Blass, W. H. Ettinger, J. B. Halter, and J. G. Ouslander, editors. New York: McGraw-Hill. 679-700.

36. Law, M. R., Morris, J. K., and Wald, N. J. 1997. Environmental tobacco smoke exposure and ischaemic heart disease: an evaluation of the evidence. Bmj 315:973-980.

37. Headrick, J. P. 1998. Aging impairs functional, metabolic and ionic recovery from ischemia-reperfusion and hypoxia-reoxygenation. J Mol Cell Cardiol 30:1415-1430.

38. Bak, M. I., Wei, J. Y., and Ingwall, J. S. 1998. Interaction of hypoxia and aging in the heart: analysis of high energy phosphate content. J Mol Cell Cardiol 30:661-672.

39. Lakatta, E. G., Sollott, S. J., and Pepe, S. 2001. The old heart: operating on the edge. Novartis Found Symp 235:172-196; discussion 196-201, 217-120.

40. Lesnefsky, E. J., Moghaddas, S., Tandler, B., Kemer, J., and Hoppel, C. L. 2001. Mitochondrial dysfunction in cardiac disease: ischemia—reperfusion, aging, and heart failure. J Mol Cell Cardiol 33:1065-1089.

41. Mariani, J., Ou, R., Bailey, M., Rowland, M., Nagley, P., Rosenfeldt, F., and Pepe, S. 2000. Tolerance to ischemia and hypoxia is reduced in aged human myocardium. J Thorac Cardiovasc Surg 120:660-667.

42. Bacon, N. C., Wappner, P., O'Rourke, J. F., Bartlett, S. M., Shilo, B., Pugh, C. W., and Ratcliffe, P. J. 1998. Regulation of the Drosophila bHLH-PAS protein Sima by hypoxia: functional evidence for homology with mammalian HIF-1 alpha. Biochem Biophys Res Commun 249:811-816.

43. Bruick, R. K., and McKnight, S. L. 2001. A conserved family of prolyl-4-hydroxylases that modify HIF. Science 294:1337-1340.

44. Wingrove, J. A., and O'Farrell, P. H. 1999. Nitric oxide contributes to behavioral, cellular, and developmental responses to low oxygen in Drosophila. Cell 98:105-114.

45. O'Farrell, P. H. 2001. Conserved responses to oxygen deprivation. J Clin Invest 107:671-674.

46. Haddad, G. G. 2000. Enhancing our understanding of the molecular responses to hypoxia in mammals using Drosophila melanogaster. J Appl Physiol 88:1481-1487.

47. Haddad, G. G., Sun, Y., Wyman, R. J., and Xu, T. 1997. Genetic basis of tolerance to O₂ deprivation in Drosophila melanogaster. Proc Natl Acad Sci USA 94:10809-10812.

48. Ma, E., Gu, X. Q., Wu, X., Xu, T., and Haddad, G. G. 2001. Mutation in pre-mRNA adenosine deaminase markedly attenuates neuronal tolerance to O2 deprivation in Drosophila melanogaster. J Clin Invest 107:685-693.

49. Donnelly, D. F., Jiang, C., and Haddad, G. G. 1992. Comparative responses of brain stem and hippocampal neurons to O2 deprivation: in vitro intracellular studies. Am J Physiol 262: L549-554.

50. Haddad, G. G., and Ma, E. 2001. Neuronal tolerance to O2 deprivation in drosophila: novel approaches using genetic models. Neuroscientist 7:538-550.

51. Nusslein-Volhard, C., and Wieschaus, E. 1980. Mutations affecting segment number and polarity in Drosophila. Nature 287:795-801.

52. White, K., Grether, M. E., Abrams, J. M., Young, L., Farrell, K., and Steller, H. 1994. Genetic control of programmed cell death in Drosophila. Science 264:677-683.

53. Artavanis-Tsakonas, S., Muskavitch, M. A., and Yedvobnick, B. 1983. Molecular cloning of Notch, a locus affecting neurogenesis in Drosophila melanogaster. Proc Natl Acad Sci USA 80:1977-1981.

54. Fortini, M. E., Skupski, M. P., Boguski, M. S., and Hariharan, I. K. 2000. A survey of human disease gene counterparts in the drosophila genome. Journal of Cell Biology 150: F23-F30.

55. Reiter, L. T., Potocki, L., Chien, S., Gribskov, M., and Bier, E. 2001. A Systematic Analysis of Human Disease-Associated Gene Sequences In Drosophila melanogaster. Genome Research 11:1114-1125.

56. Spradling, A. C., Stern, D., Beaton, A., Rhem, E. J., Laverty, T., Mozden, N., Misra, S., and Rubin, G. M. 1999. The Berkeley Drosophila Genome Project gene disruption project: Single P-element insertions mutating 25% of vital Drosophila genes. Genetics 153:135-177.

57. Williamson, J. R., Schaffer, S. W., Ford, C., and Safer, B. 1976. Contribution of tissue acidosis to ischemic injury in the perfused rat heart. Circulation 53:I3-14.

58. Greenspan, R. J. 2001. The flexible genome. Nat Rev Genet 2:383-387.

59. Miquel, J., Lundgren, P. R., Bensch, K. G., and Atlan, H. 1976. Effects of temperature on the life span, vitality and fine structure of Drosophila melanogaster. Mechanisms of Ageing & Development 5:347-370.

60. Braunwald, E., editor. 1992. Heart disease. Philadelphia: W B Saunders.

61. Krishnan, S. N., Sun, Y. A., Mohsenin, A., Wyman, R. J., and Haddad, G. G. 1997. Behavioral and electrophysiologic responses of Drosophila melanogaster to prolonged periods of anoxia.

62. U.S. Patent Application No. 60/270,277, filed Feb. 20, 2001, entitled Screening Procedure for Genes or Agents Affecting the Heart.

63. U.S. Patent Application Ser. No. 10/077,670, filed Feb. 15, 2002, entitled Methods of Screening for Genes Affecting Cardiac Function.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

What is claimed is:

1 A method of screening for a gene affecting cardiac function after or during hypoxia or anoxia, comprising the steps of:
a exposing an adult Drosophila to conditions able to induce cardiac hypoxia or anoxia;
b imaging the heart of said Drosophila;
c measuring the movements of the heart in the image; and
d analyzing the measurements of said movements; and
e identifying a gene affecting the cardiac function of said Drosophila.

2 The method of claim 1, further comprising exposing said Drosophila to change in temperature.

3 The method of claim 1, wherein said gene affects an age-related change in said cardiac function.

4 The method of claim 1, wherein said Drosophila is a Drosophila melanogaster.

5 The method of claim 1, wherein said imaging said heart comprises positioning said Drosophila under a microscope so that the light beam of said microscope is generally perpendicular to the frontal plane of said Drosophila and is directed on the heart of said Drosophila.

6 The method of claim 5, wherein at least one contrast enhancement means is combined with said microscope to improve said image of said heart.

7 The method of claim 5, wherein said microscope is a fluorescence microscope and wherein said Drosophila expresses a fluorescent protein in said heart able to be detected by said fluorescent microscope.

8 The method of claim 7, wherein said fluorescent protein is fluorescent green protein.

9 The method of claim 1, wherein said movements are the movements of the walls of said heart.

10 The method of claim 1, wherein said analyzing said measurements corresponds to determining the heart rate.

11 The method of claim 1, wherein said measuring said movement is obtained using movement detection software.

12 The method of claim 1, wherein said analyzing said measurements comprises comparing said measurements to a control set of data.

13 The method of claim 1, wherein said gene has a mutation.

14 The method of claim 13, wherein said mutation causes a change in expression of said gene.

15 The method of claim 14, wherein said change in expression of said gene causes an age-related change in said cardiac function.

16 The method of claim 13, wherein said mutation causes an age-related change in cardiac function.

17 A method of screening for agents affecting cardiac function after or during hypoxia or anoxia, comprising the steps of:
a exposing an adult Drosophila to conditions able to induce cardiac hypoxia or anoxia;
b exposing said Drosophila to an agent;
c imaging the heart of said Drosophila;
d measuring the movements of said heart in said image;
e analyzing the measurements of said movements; and
f identifying an effect of said agent on the cardiac function of said Drosophila by comparing said analysis to a control.

18 The method of claim 17, further comprising exposing said Drosophila to change in temperature.

19 The method of claim 17, wherein the effect of said agent on age-related changes in the cardiac function is determined.

20 The method of claim 17, wherein said measurements are compared to a control set of data.

21 The method of claim 17, wherein the movements are movements of the walls of said heart.

22 The method of claim 17, wherein said analyzing comprises determining the heart rate of said Drosophila.

23 The method of claim 17, wherein said measuring is obtained using movement detection software.

24 The method of claim 17, wherein said Drosophila is a Drosophila melanogaster.
